(19) United States
(12) Patent Application Publication
(10) Pub. No.: US 2023/0236168 A1
(43) Pub. Date: Jul. 27, 2023

(54) TEST SYSTEM

(71) Applicant: Senseair AB, Delsbo (SE)

(72) Inventors: Mathias Granstam, Västerås (SE); Karl-Adam Hessman Pettersson, Västerås (SE); Niclas Holmqvist, Västerås (SE)

(21) Appl. No.: 18/002,565

(22) PCT Filed: Jun. 29, 2021
(86) PCT No.: PCT/SE2021/050647

§ 371 Date: Dec. 20, 2022

(30) Foreign Application Priority Data
Jul. 2, 2020 (EP) 2050823-0

(51) Int. Cl. G01N 33/497; G01N 33/00

(52) U.S. Cl. CPC G01N 33/4972; G01N 33/007; G01N 2033/0072

(57) ABSTRACT

A system for testing alcohol breath analyzers, wherein the system comprises an alcohol breath gas generation device, a test chamber, a reference measuring device, a control and registration unit, and a distribution unit. The invention also relates to methods of testing alcohol breath analyzers.

### FIELD OF INVENTION

The present invention relates to a system for detection and quantification of substances in a gas mixture, as well as a system and method for generating a gas mixture. The present invention also relates to methods of testing performance of alcohol breath analyzer units.

### BACKGROUND

Drunk driving is a global problem that accounts for thousands of lives annually in US alone, similar numbers have been recorded in Europe. Most countries today have a limit for alcohol content in the breath allowed during driving. To enforce this limit alcohol breath analyzers are important tools. The current state of the art alcohol breath analyzers requires a forced exhalation received by a mouthpiece. Recently there has been a development for a novel type of alcohol breath analyzers that do not require a mouthpiece or a forced exhalation. Such alcohol breath analyzers are more user friendly and therefore more likely to be accepted by the general public. One of the challenges with such a device or system without a mouthpiece is that the amount of available gas from the exhalation is much lower. The detection limit of the analyzer therefore needs to be lower than for current devices, they also must be reliable. An alcohol breath analyzer also needs to be able to detect the breath alcohol content at various environmental conditions. Therefore, a test system further needs to be able to test alcohol breath analyzers at different conditions, such as different temperatures and different relative humidities.

US 2018/0252699 discloses a metrological bench for calibrating breath alcohol testers, the metrological bench is adapted to carry out a method involving delivering to the tester a sample of gas that varies in terms of ethanol concentration, CO₂ concentration, flow rate, pressure and temperature in a manner equivalent to the variances exhibited by a human.

US 2011/0107813 discloses a breath test analyzer with vapor including ethyl alcohol including a heated thermal mass to heat an inlet passage.

J. Ljungblad “High performance breath alcohol analysis” Malardalen University Press Dissertations No. 240 2017 discloses alcohol breath analyzers designed for operations without a mouthpiece. Experiments show that given enough time and analyzer resolution, passive alcohol detection systems are feasible.

There is a need for improvement of the systems and methods that generates a gas mixture that mimics a human exhalation, wherein the gas mixture can comprise a controlled amount of alcohol. The system could be used to the test the performance of alcohol breath analyzers Another need in the area is a system that enables testing of alcohol breath analyzers at varying environmental conditions.

### SUMMARY

The object of the invention is to obtain an improved system and method for generation of gas that mimics the human exhalation, wherein the system can be used to the test the performance of alcohol breath analyzers. This is achieved by the system in claim 1 and the method in claim 13.

In a first aspect there is a system for testing at least one alcohol breath analyzer comprising:
An alcohol breath gas generation device comprising at least one liquid column. The gas generation device comprises a gas inlet arranged at the lower part of the gas generation device and a gas outlet arranged at the upper part of the gas generation device. Gas enters the gas generation device at the gas inlet and exits the gas generation device at the gas outlet, and the liquid in the liquid column comprises water;
A test chamber in fluid communication with the alcohol breath gas generation device. The test chamber is arranged to receive at least one alcohol breath analyzer;
A reference measuring device in fluid communication with the alcohol breath gas generation device;
A control and registration unit arranged to control the parts of the alcohol breath analyzer system and to register the testing results; and
A distribution unit in fluid communication with the gas generation device. The distribution unit arranged to distribute gas from the gas generation device to one of three gas flow paths: a first gas flow path arranged to distribute gas from the gas generation device to the test chamber, a second gas flow path arranged to distribute gas from the gas generation device to the reference measuring device, and a third gas flow path arranged to distribute gas from the gas generation device 101 to an opening to the ambient air.

In one embodiment the system comprises valve means arranged to shift the gas flow between the three gas flow paths: the first gas flow path, the second gas flow path, and the third gas flow path.

In one embodiment the third gas flow path is arranged to distribute gas from the gas generation device to an outlet.

In one embodiment the test system comprises a first valve arranged at the inlet at the gas generation device, arranged to regulate the gas flow into the gas generation device, a second valve arranged in between the gas generation device, and the distribution unit, the second valve is arranged to regulate the gas flow from the gas generation device to the distribution unit, a third valve, a fourth valve, and a fifth valve arranged at the distribution unit. The third valve is arranged to regulate the gas flow into the first gas tube, the fourth valve is arranged to regulate the gas flow into the third gas tube, and the fifth valve is arranged to regulate the gas flow into the second gas tube. The first valve, the second valve, and the third valve are arranged to control the gas flow in the first gas flow path. The first valve, the second valve, and the fifth valve are arranged to control the gas flow in the second gas flow path. The first valve, the second valve, and the fourth valve are arranged to regulate the gas flow in the third gas flow path.

In one embodiment the inner diameter of the valves is 0.5-2 cm.

In one embodiment the test system comprises at least a first and a second heater arranged to heat the system so that the temperature is higher at the distribution unit than at the gas generation device.

In one embodiment the gas generation device has a volume of 3000-7000 cm³.

In one embodiment the liquid column has a volume of 2000-3000 ml.

In one embodiment the gas generation device further comprises a dead space, and wherein the volume of the dead space is 0.1-3.5 L.

In one embodiment the test system further comprises a device for changing alcohol breath analyzer arranged in the test chamber.

In one embodiment the device for changing alcohol breath analyzer comprises a revolver mechanism.

In one embodiment the test system comprises more than one gas generation devices, such as two gas generation devices or three.

In a second aspect there is a method of testing performance of at least one alcohol breath analyzer using a system that comprises an alcohol breath gas generation device, a test chamber in fluid communication with the alcohol breath gas generation device, the test chamber is arranged to receive at least one alcohol breath analyzer, a reference measuring device in fluid communication with the alcohol breath gas generation device, a control and registration unit arranged to control the parts of the alcohol breath analyzer system, and a distribution unit in fluid communication with the gas generation device. The distribution unit arranged to distribute gas from the gas generation device to one of three gas flow paths: a first gas flow path arranged to distribute gas from the gas generation device to the test chamber, a second gas flow path arranged to distribute gas from the gas generation device to the reference measuring device, and a third gas flow path arranged to distribute gas from the gas generation device to an outlet. The method of testing comprises the steps of:
receiving input values for temperature, relative humidity, alcohol concentration to the system;
flushing the system first via the second gas flow path, and then via the first gas flow path;
the reference unit measuring the alcohol, water, and CO₂ concentration of the gas generated by the gas generation device; and
the at least one alcohol breath analyzer unit arranged in the test chamber measuring alcohol, water and CO₂ concentration.

In one embodiment of the method the system further comprises a first valve arranged at the inlet at the gas generation device, a second valve arranged in between the gas generation device and the distribution unit, a third valve, a fourth valve, and a fifth valve arranged at the distribution unit. The valves are arranged to control the gas flow in the system and the method comprises the steps of:
opening the first valve to provide gas to an endpoint of one of the three gas flow paths: the first gas flow path; the second gas flow path; or the third gas flow path, in a sequence starting with the outermost downstream valve and consecutively open the plurality of valves of one of the first gas flow path; the second gas flow path; or the third gas flow path, in the upstream direction finishing with the first common valve; and
ending the provision of gas to an endpoint by closing the valves of one of the first gas flow path; the second gas flow path; or the third gas flow path starting with the first common valve and consecutively close the plurality of valves in the flow path in the downstream direction finishing with the valve closest to the endpoint, i.e. the third valve for the first gas flow path, the fifth valve for the second gas flow path, and the fourth valve for the third gas flow path.

In one embodiment of the method one of the third valve, fourth valve, and fifth valve is opened first. After the opening of one of the third valve, fourth valve, or fifth valve the second valve is opened, and after the opening of the second valve the first valve is opened.

In one embodiment of the method it comprises the substeps of:
flushing the second gas flow path, by opening the fourth valve, followed by the second valve, and last the first valve, after a pre-determined time period the first valve is closed first, followed by the second valve, and last the fourth valve is closed; and
flushing via the first gas flow path, by opening the third valve, followed by the second valve, and last the first valve, after a pre-determined time period the first valve is closed first, followed by the second valve, and last the third valve is closed;
opening the fifth valve, followed by the second valve, and last the first valve, after a pre-determined time period the first valve is closed first followed by the second valve, and last the fifth valve; and
opening the third valve, followed by the second valve, and last the first valve, after a pre-determined time period the first valve is closed first followed by the second valve and last the third valve.

In one embodiment of the method the step of measuring alcohol, water and CO₂ concentration with the at least one alcohol breath analyzer unit arranged in the test chamber is repeated at least once. In one embodiment the step is repeated ten times.

### LIST OF FIGURES

FIG. 1a is a schematic illustration of one embodiment according to the invention;

FIG. 1b is a schematic illustration of a gas flow path chart according to the invention;

FIG. 2 is a schematic illustration of one embodiment according to the invention;

FIG. 3 is a schematic illustration of one embodiment according to the invention;

FIG. 4 is a schematic illustration of a method according to the invention;

FIG. 5 is a schematic illustration of a method according to the invention; and

FIGS. 6a and b are schematic illustrations of methods according to the invention.

### DETAILED DESCRIPTION

Terms such as “top”, “on top”, “bottom”, upper”, lower”, “below”, “above” etc. are used merely with reference to the geometry of the embodiment of the invention shown in the drawings and/or during normal operation of the described system and its components are not intended to limit the invention in any manner.

The aim of an alcohol breath analyzer is to measure (or test) the alcohol concentration in breath from a human. The output (result) from such a test can be used for example to determine whether the person can drive a car or not depending on the concentration of alcohol in the breath. For such a task an alcohol breath analyzer needs to be reliable and measure the correct alcohol concentration in the breath. Therefore, the performance, i.e. the margin of error for such measurements, of alcohol breath analyzers are tested in a test system. A test system may be arranged to test the performance of individual alcohol breath analyzers.

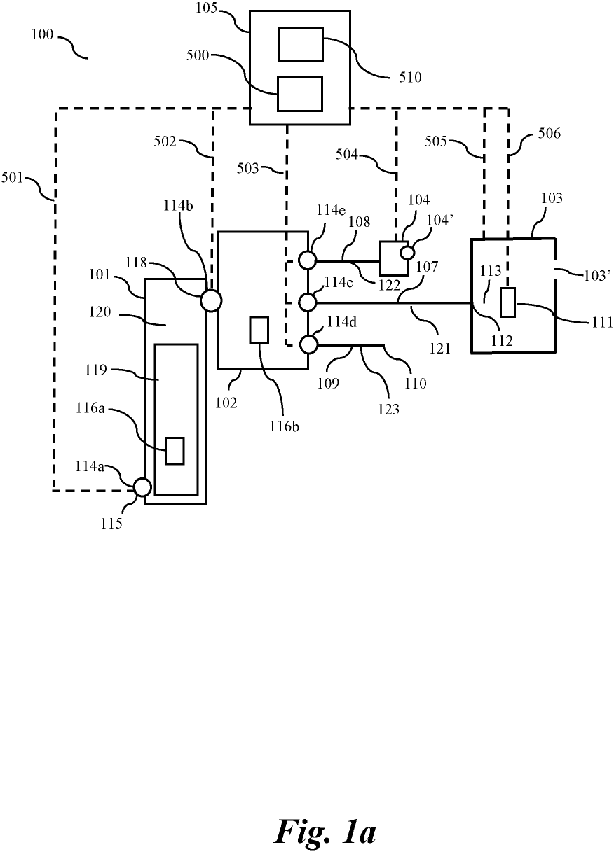

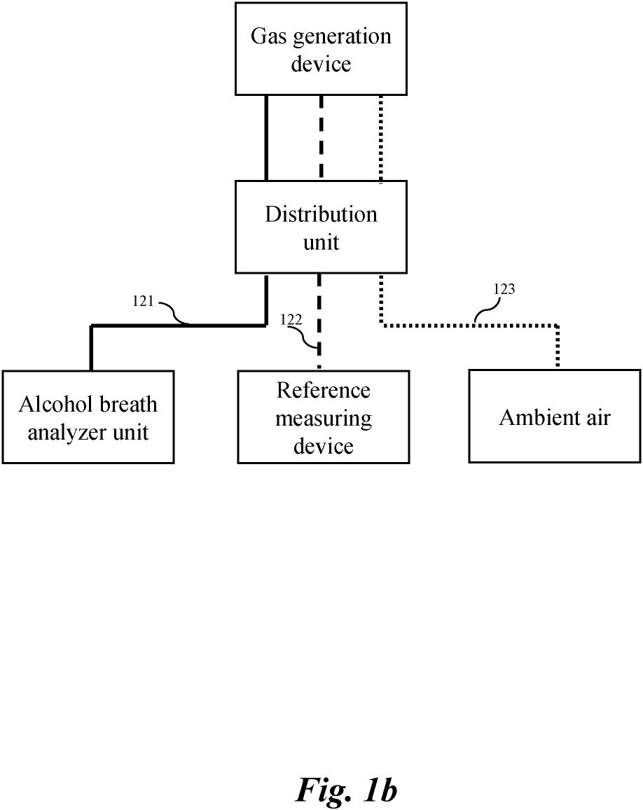

In one aspect of the invention there is a system 100 for testing alcohol breath analyzer units 111. Such a system is schematically illustrated in FIG. 1a. The system 100 comprises an alcohol breath gas generation device 101, a distribution unit 102, a test chamber 103, a reference measuring device 104, and a control and registration unit 105. The alcohol breath gas generation device 101 is arranged to generate a gas mixture comprising alcohol, or a gas mixture without alcohol. The gas generation procedure is explained in further detail below. The gas mixture may be a mixture of for example air, carbon dioxide (CO₂), water, and alcohol vapor so that it corresponds to a human exhalation containing alcohol vapor. The alcohol breath gas generation device 101 is in fluid communication with the distribution unit 102 that is arranged downstream the alcohol breath gas generation device 101. The distribution unit 102 is arranged to distribute the gas generated by the alcohol breath gas generation device 101 further downstream the system 100, via a first gas flow tube 107, a second gas flow tube 108, and a third gas flow tube 109. The first gas flow tube 107 is arranged to distribute gas from the distribution unit 102 to the test chamber 103, the second gas flow tube 108 is arranged to distribute gas from the distribution unit 102 to the reference measuring device 104, and the third gas flow tube 109 is arranged to distribute gas from the distribution unit 102 to a waste outlet 110. The system 100 provides for three functional gas flow paths, schematically illustrated in the gas flow path chart of FIG. 1b. The first gas flow path 121 functionally provides the gas generated in the gas generator to the alcohol breath analyzer unit, via the distribution unit. The second gas flow path 122 functionally provides the gas generated in the gas generator to the reference measuring device, via the distribution unit. The third gas flow path 123 functionally provides means to flush the system by providing an opening to ambient air, via the distribution unit.

The gas generation device 101, the distribution unit 102, and the first gas flow tube 107 forms a first gas flow path 121 leading from the gas generation device 101 to the test chamber 103. The gas generation device 101, the distribution unit 102, and the second gas flow tube 108 forms a second gas flow path 122 leading from the gas generation device 101 to the reference measuring device 104. The gas generation device 101, the distribution unit 102, and the third gas flow tube 109 forms a third gas flow path 123 leading from the gas generation device 101 to the waste outlet 110. The third gas flow path 123 may, as depicted in FIG. 1a end in a separate outlet 110 from the distribution outlet 102. Alternatively, representing different embodiments an opening to the ambient air is provided in the test chamber 103, opening 103′, or in connection with the reference instrument, reference valve 104′ that can provide an opening to the ambient air. The waste outlet 110, the test chamber 103, and the reference measuring device 104 are all arranged downstream the distribution unit 102. Hence, the alcohol breath gas generation device 101 is during use of the system 100 in fluid communication with the distribution unit 102 that is in fluid communication with the test chamber 103, the reference measuring device 104, and the waste outlet 110. In this way gas that is generated by the gas generation device 101 reach the gas distribution unit 102 where it is distributed to one of the three flow tubes (i.e. the first gas flow tube 107, the second gas flow tube 108, or the third gas flow tube 109). The gas that enters the first gas flow tube 107 travels from the gas distribution unit 102 to the test chamber 103, the gas that enters the second gas flow tube 108 travels from the gas distribution unit 102 to the reference measuring device 104, and the gas that enters the third gas flow tube 109 travels from the gas distribution unit 102 to the waste outlet 110.

During use of the test system 100 at least one alcohol breath analyzer unit 111 is placed in the test chamber 103. The test system 100 is arranged to provide a gas mixture that the at least one alcohol breath analyzer unit 111 is exposed to. The alcohol breath gas generation device 101 is arranged to generate said gas mixture during use of the test system 100. The gas mixture can be any mixture of air, carbon dioxide, water, and/or alcohol. In other embodiments the alcohol in the mixture may be exchanged for another substance such as for example methanol or acetone. In such embodiments the at least one alcohol breath analyzer unit 111 may be exchanged for an analyzer unit configured to measure the substance provided in the gas mixture. Additionally, the reference measuring device may be a reference measuring device configured to measure the substance provided in the gas mixture.

In case that the gas mixture comprises alcohol the concentration of alcohol is known, the gas mixture may further have a known concentration of carbon dioxide and/or water.

The system 100 may comprise one or more heaters, such as a first heater 116a and a second heater 116b arranged to heat, or maintain a predetermined temperature of, the gas mixture and/or other components or parts of the system 100. Keeping the gas mixture at a controlled temperature may be important in order to not change the composition of the gas, i.e. if the temperature of the gas varies the composition of the gas mixture may vary. It may for example be important that the temperature of the gas in the system 100 is above the temperature of the dew point of the gas. During use of the system 100 the at least one alcohol breath analyzer unit 111 is arranged to be exposed to the gas mixture. Such an exposure can be used to test the performance of the at least one alcohol breath analyzer unit 111. If the at least one alcohol breath analyzer unit 111 is arranged to be exposed to a gas mixture without alcohol, the at least one alcohol breath analyzer unit 111 may be tested at a zero-level alcohol content. The at least one alcohol breath analyzer unit 111 is arranged to measure the alcohol content, i.e. the alcohol concentration, and/or the carbon dioxide content of the gas with known alcohol concentration in order to test its performance. The at least one alcohol breath analyzer unit 111 may comprise a device for measuring and controlling temperature.

Gas flow tubes 107; 108; 109 are arranged to distribute gas through the system 100. The gas flow tubes 107; 108; 109 may be fabricated in for example a stainless material or a polymer such as polytetrafluoroethylene (PTFE). The exit 112 is arranged to allow gas from the second gas flow tube 107 to enter the test chamber 103. At the exit 112 the inner cross-section of the gas flow tube 107 may be 1-4 cm, or ½″, so that it mimics the size of an open mouth. Furthermore, between the exit 112 and the alcohol breath analyzer unit 111 there is a gas cloud space 113. The gas cloud space 113 may be arranged to be empty so that no item is placed in between the exit 112 and the alcohol breath analyzer unit 111 to interfere with the gas flow from the exit 112 to the alcohol breath analyzer unit 111. In this way the gas flow may mimic an exhalation from a person. The gas cloud space 113 is a certain volume in the test chamber 103 that has a certain distance, for example 0-30 cm, so that the distance between the exit 112 and the alcohol breath analyzer unit 111 is for example 0-30 cm.

Valves 114a-e are arranged to regulate the flow of gas in the system 100 during use of the system 100. A first valve 114a is arranged at the inlet 115 to the alcohol breath gas generation device 101. A second valve 114b is arranged in between the gas generation device 101 and the distribution unit 102. The distribution unit 102 further comprises three additional valves: a third valve 114c, a fourth valve 114d, and a fifth valve 114e arranged at the first gas flow tube 107, the second gas flow tube 108, and the third gas flow tube 109, respectively. The valves 114a-e may be arranged to enable so that the system 100 has the same, or essentially the same, internal pressure at all parts of the system 100. This may for example be achieved by valves 114a-e with an inner diameter of 0.5-2 cm, or ¼″. It may also be achieved using pneumatic valves inside the system 100, hence that the valves 114a-e are pneumatic.

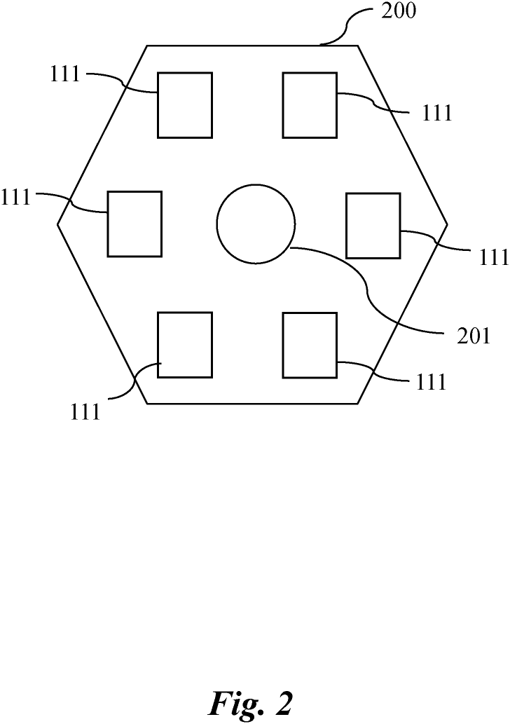

At least one alcohol breath analyzer unit 111 is arranged to be placed in the test chamber 103 during use of the test system 100. In other embodiments the test chamber 103 may hold more than one alcohol breath analyzer unit 111, such as two or more, or three or more, or four or more alcohol breath analyzer units 111, during use of the test system 100. In one embodiment the test chamber 103 comprises a device for changing alcohol breath analyzer 200, schematically illustrated in FIG. 2. The device 200 may comprise a revolver mechanism. In embodiments where the test chamber 103 comprises a device for changing alcohol breath analyzer 200 the test chamber may hold several alcohol breath analyzer units 111 that may be tested sequentially so that the device for changing alcohol breath analyzer 200 is arranged to change the alcohol breath analyzer unit 111 that is tested by the system 100. The device for changing alcohol breath analyzer 200 may comprise an axis 201 so that it can be rotated in order to change the alcohol breath analyzer unit 111 that is tested. During testing an alcohol breath analyzer 111 is positioned in front of the gas exit 112. The device for changing alcohol breath analyzer 200 may be positioned so that the distance between the alcohol breath analyzer 111 and the gas exit 112 is the same and remain constant during the testing sequence for all alcohol breath analyzers 111. Hence, the device for changing alcohol breath analyzer 200 may be arranged to maintain an alcohol breath analyzer unit 111 at a predetermined distance from the gas exit 112 during a predetermined time period, during which the alcohol breath analyzer unit 111 may be arranged to be exposed to a gas mixture.

In one embodiment the test chamber 103 is a climate chamber so that the test chamber 103 may be arranged to change and/or control the temperature and/or relative humidity (RH) inside the test chamber 103. In an embodiment where the test chamber 103 is a climate chamber the at least one alcohol breath analyzer unit 111 may be arranged to be tested at different relative humidities and/or temperatures, such as at various environmental conditions of ambient temperature and humidity. In one embodiment the test chamber 103 is arranged to provide temperatures between -40° C. to +120° C., and/or relative humidities (RH) between 10 % and 95 %. The test chamber 103 comprises one opening 103′ towards the ambient air. Such an opening 103′ may enable that the gas that enters the test chamber 103 at the exit 112 does not stay inside the test chamber 103 and build up a background level but instead exit the test chamber 103 at the opening 103′. The opening 103′ may be realized by for example an opening in the wall of the test chamber 103, a filter, a door that can be opened, a valve, etc. The opening 103′ may also be controlled by for example the control and registration unit 105.

The waste outlet 110 is arranged downstream the distribution unit 102 and the alcohol breath gas generation device 101. Hence, the distribution unit 102 may be flushed with gas that can exit the system 100 at the waste outlet 110. The waste outlet 110 may be open to the ambient air. In such way the system 100 may be flushed with gas in between testing sequences. Flushing the system 100 via the third gas flow path 123 so that the gas exits the system 100 at the outlet 110 may prevent a background level from building up in the test chamber 103 during the flushing.

The system 100 may be arranged to be heated in order to produce gas with a composition that mimics human breath and/or in order to avoid condensation of the gas inside the system 100. In order to heat the system 100, it may comprise heaters: a first 116a and a second 116b heater. In one embodiment the first heater 116a is arranged at the alcohol breath gas generation device 101, and the second heater 116b is arranged at the distribution unit 102. The gas flow tubes 107; 108; 109 may also be heated, the gas flow tubes 107; 108; 109 may be heated in sections so that the temperature of the gas flow tubes 107; 108; 109 is lower downstream in the system 100 than upstream, in such way condensation of gas inside the gas flow tubes 106; 107; 108; 109 may be avoided. The temperature of the parts of the system 100 may be higher upstream in the system 100 than downstream. The temperature of the gas flow tubes 107; 108; 109 may be stable over time. That the temperatures of the gas flow tubes 107; 108; 109 are stable may enable that the composition of the gas does not vary. In one embodiment the gas distribution unit 102 is arranged to be heated to a temperature of 30-40° C. during use of the system 100, for example the temperature of the gas generation device 101 may be 34° C., the temperature of the distribution unit 102 may be 36° C., and the temperature of the gas flow tubes 107; 108; 109 may be 38° C.

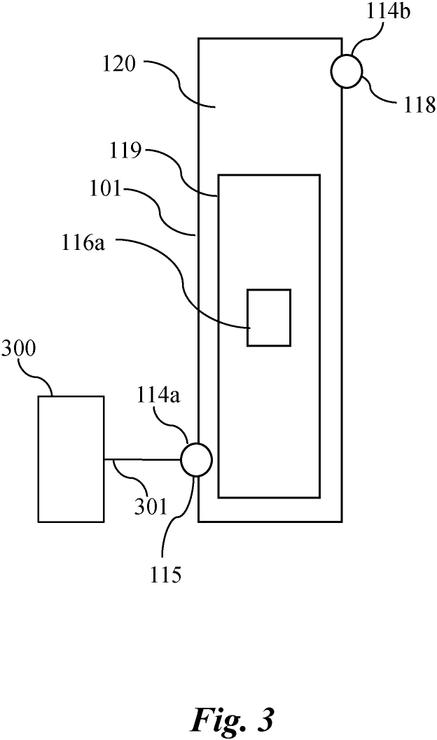

The alcohol breath gas generation device 101 in the system 100 is arranged to generate a gas mixture comprising water, air, carbon dioxide and alcohol. It may also generate a gas composition without alcohol. An alcohol gas generation device 101 is schematically illustrated in FIG. 3. The alcohol gas generation device 101 comprises a gas inlet 115 at which a first valve 114a is arranged, the first valve 114a is positioned at the lower part of the alcohol breath gas generation device 101, and a gas outlet 118 arranged at the upper part of the alcohol breath gas generation device 101 where a second valve 114b is arranged. Gas is arranged to enter the alcohol breath gas generation device 101 at the first valve 114a and pass through a liquid column 119 to a dead space 120 before it exits the alcohol breath gas generation device 101 at the gas outlet 118. The gas in the dead space 120 may have a different composition than the gas that enters the gas generation device 101 at the inlet 115. The composition of the gas in the dead space 120 may depend on the composition of the liquid column 119. The liquid column 119 is arranged to comprise a liquid, the liquid may be water or a mixture of water and alcohol. During use of the system 100 gas is arranged to pass through the liquid column 119 so that a gas mixture comprising alcohol is generated in the dead space 120, the gas mixture generated in the dead space 120 may also be a gas mixture that does not comprise alcohol. The alcohol concentration in the gas mixture is related to the alcohol concentration in the liquid column 119, hence by varying the alcohol concentration in the liquid column 119 the alcohol concentration in the gas mixture may be varied. In one embodiment the liquid column 119 is arranged to comprise water without alcohol. In such an embodiment when gas passes through the liquid column 119 the gas mixture generated in the dead space 120 does not comprise alcohol. The liquid in the liquid column 119 may be arranged to be saturated with CO₂. During use of the system 100 gas is arranged to enter the alcohol breath gas generation device 101 at the first valve 114a, the gas may be in the form of air, or CO₂, or N₂, or any mixture of those. The gas generation device 101 may be connected to a gas tank 300 at the first valve 114a via a gas inlet tube 301. The volume and the height of the liquid column 119 as well as the volume of the dead space 120 may affect the gas generation. During use of the test system 100 and/or the alcohol gas generation device 101 gas is arranged to bubble through the liquid column 119. Bubbling gas through the liquid column 119 may lead to that:
the liquid in the liquid column 119 is saturated, or reached a constant steady state concentration of CO₂, with the gas, e.g. saturated with CO₂; and
an equilibrium concentration of both water vapor and possible alcohol, air and carbon dioxide can be reached in the dead space 120.

During use of the system 100 gas in the dead space is arranged to travel further downstream the system 100 to reach either the outlet 110, the test chamber 103, or the reference measuring device 104. In one embodiment the volume of the dead space 120 is 0.1-3.5 L. A large volume of the dead space 120 may enable that aerosols formed in the alcohol gas generation device 101 can fall out in the dead space 120 and thus not travel with the gas further in the system 100. The absence of aerosols in the gas mixture may enable a more stable gas composition, i.e. that the concentrations of the gas components does not vary.

In one embodiment the gas flow in the system 100 may be 2 L/s, or 20 L/min, or around 0.5 L/s. The gas flow level may depend on the volume of the liquid column 119, the height of the liquid column 119, the volume of the dead space 120, etc. In one embodiment the volume of the liquid 119 is 2000-3000 ml and the height of the liquid column 119 is 200-400 mm. The gas flow in the system 100 may be measured as input flow.

In one embodiment the liquid in the liquid column 119 is arranged to be mixture of water and alcohol, and the mixture is arranged to be heated to 34° C. and bubbled with air, or air enriched with carbon dioxide (CO₂). The gas generation device 101 may be arranged to generate a gas mixture in the dead space 120 comprising alcohol, water, and CO₂.

In one embodiment the alcohol breath gas generation device 101 comprises more than one liquid column 119, such as two liquid columns, or three liquid columns or more. Each liquid column 119 may have its own inlet 115, outlet 118, heater 116 and dead volume 120. More than one liquid column 119 enables that the test system 100 may be arranged to generate gas mixtures with different concentrations without changing the liquid in the liquid column 119. Hence, each liquid column 119 may be arranged to comprise a different liquid composition that for example have different alcohol concentrations so that gas formed in the dead volume 120 above respective liquid column 119 have different compositions.

The gas that enters the liquid column 119 at the gas inlet 115 may be arranged to be dispersed so that it is in the form of small bubbles, i.e. with a diameter of 0.3-6.5 mm. Small bubbles may facilitate a more homogenous mixture of the gas with the liquid in the liquid column 119 so that the different concentrations may reach equilibrium before the gas reach the dead volume 120.

The reference measuring device 104 is arranged downstream the distribution unit 102 and the gas generation device 101. The reference measuring device 104 is arranged to measure the composition of the gas generated by the gas generation device 101. During a test sequence or method of testing the reference measuring device 104 may be arranged to verify the composition of the gas. The reference measuring device 104 may be a reference instrument based on IR detection. The reference measuring device 104 may comprise a reference valve 104′ that can be opened to the ambient air. The reference valve 104′ may enable that the reference measuring device 104 can be arranged to flush itself during use of the test system 100. The measurements by the reference measuring device 104 may be used for comparison with the measured values from the alcohol breath analyzer unit 111. During a test sequence or method of testing, both the reference measuring device 104 and the alcohol breath analyzer unit 111 are arranged to be exposed for gas with essentially the same composition. Both the reference measuring device 104 and the alcohol breath analyzer unit 111 measures the alcohol concentration in the gas and the results may be compared for example to determine the performance of the alcohol breath analyzer unit 111. The reference measuring device 104 may be an instrument that fulfill the requirements on evidential instruments set for example in the Scandinavian countries. It may be a commercially available instrument that is verifiable. The reference measuring device 104 may be based on IR detection and operate according to Beer Lambert’s law, in such case in order to verify the reference measuring device 104 it is only necessary to verify that the relative attenuation at each of the filter wavelengths is the same as it was at the time for calibration. Such a reference measuring device 104 may be an evidential breath analyzer, for example Evidenzer, Nanopuls AB, Uppsala, Sweden, with a documented measuring accuracy and precision exceeding that of standard breath analyzers. The accuracy and precision of the reference measuring device 104 may repeatedly be verified by gravimetric methodology.

The test system 100 according to the invention is controlled via the control and registration unit 105 which also is configured to collect test data. The control and registration unit 105 typically comprises a processor 500 and at least one storage unit 510 for storing the program sequences and collected data and an I/O-unit for communication with a multi-purpose computer, for example. The control and registration unit 105 is functionally connected to the first valve 114a via a first functional signal pathway 501, the second valve 114b via a second functional pathway 502, the distribution unit 102 comprising the third 114c, fourth 114d, and fifth valve 114e, via a third functional signal pathway 503, the reference measuring device 104 via a fourth functional signal pathway 504, the test chamber 103 via a fifth functional signal pathway 505, and to the at least one alcohol breath analyzer unit 111 via a sixth functional signal pathway 506. As realized by the skilled person the hardware of the control and registration unit 105 and the connection to other units in the test system 100 may be realized in a number of ways, for example the connections being directly wired, via a bus-system or wireless. Similarly, the architecture of a control and registration unit 105 may vary and the described units should be regarded as functional units. Suitable multi-purpose control and registration units suitable for the test system according to the invention are commercially available, for example.

A test system 100 according to the invention may enable at least one alcohol breath analyzer unit 111 to be tested using gas with an alcohol concentration that has a variation of less than 0.5 µg/L, or less than 0.1 µg/L. For a test system to be reliable it is important that the concentration of the gas composition, for example the alcohol concentration in the gas does not show a large variation. A test system 100 according to the invention, or a gas generation device 101 may generate a gas composition wherein the alcohol concentration is 0-5 mg/L, or 0-2.5 mg/L.

The valves 114a-e in the system 100 can be automated in order to regulate the flow of gas through the system 100. The control and registration unit 105 may be configured to provide such a regulation of the valves 114a-e.

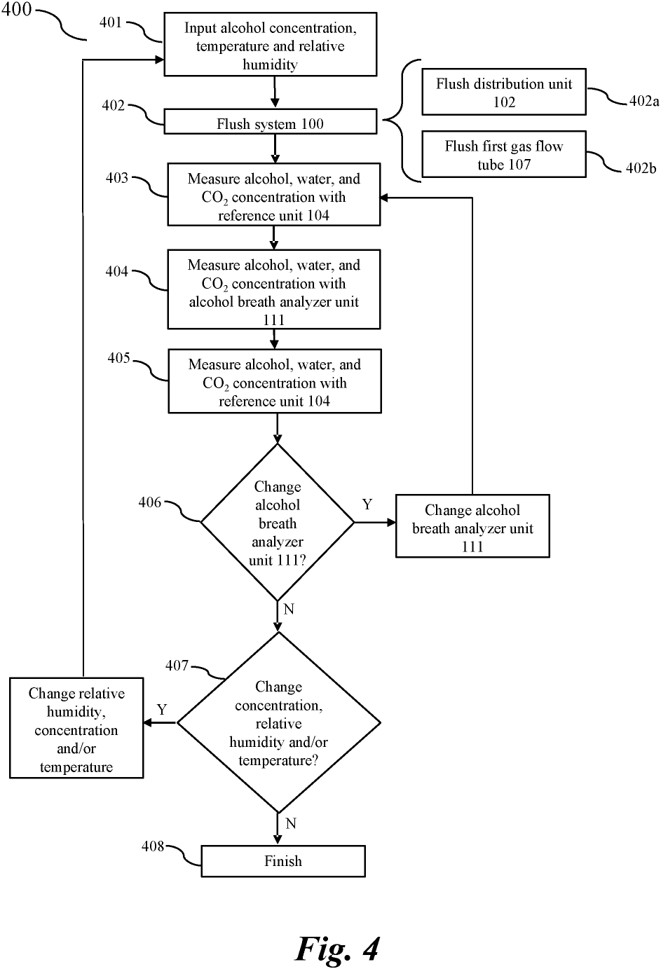

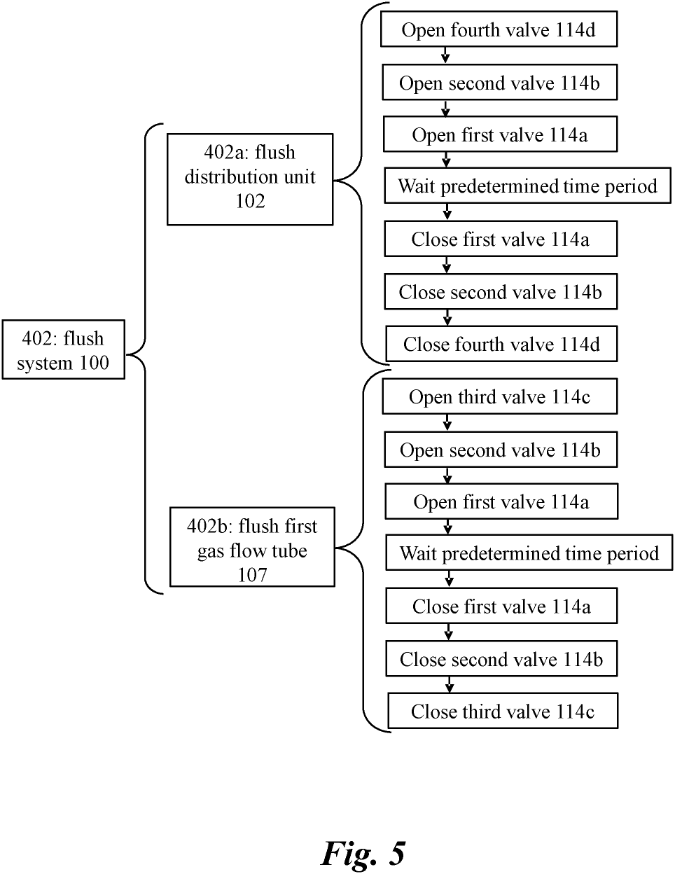

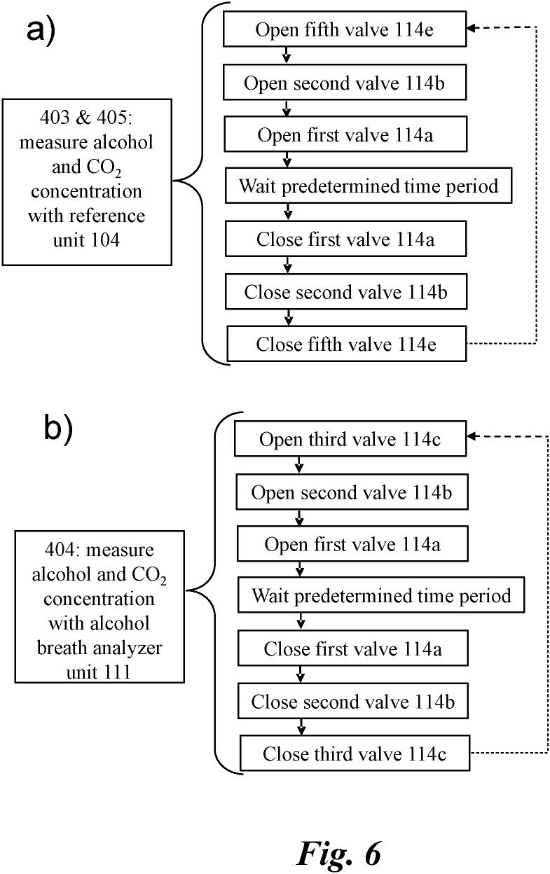

The at least one alcohol breath analyzer unit 111 may be tested for performance by the system 100, e.g. how accurate the alcohol breath analyzer unit 111 can measure the concentrations of alcohol, CO₂, and possible other gases in a gas mixture. Such a test is performed by exposing the at least one alcohol breath analyzer unit 111 for gas generated by the system 100 and/or gas generation device 101. The at least one alcohol breath analyzer unit 111 is exposed to the gas in a method of testing 400, illustrated in FIG. 4. During the method of testing 400 the at least one alcohol breath analyzer unit 111 is arranged in the test chamber 103. Prior to starting a method of testing 400 all valves 114a-e in the system 100 are closed. The method of testing 400 comprises the following steps:
401: input values for temperature, relative humidity, alcohol concentration to the system 100;
402: flush system 100, step 402 comprises the substeps:
   402a: flush system via the third gas flow path 123, this step may ensure that the distribution unit 102 comprises the alcohol concentration inputted in step 401. To perform step 402a the fourth valve 114d is opened first, followed by the second valve 114b, and last the first valve 114a. Once the first valve 114a is opened gas is transported in the system 100 via the third gas flow path 123 and exits the system at the outlet 110. After a pre-determined time period the first valve 114a, second valve 114b, and fourth valve 114d that are open are closed again in the reverse order, i.e. the first valve 114a is closed first, followed by the second valve 114b, and last the fourth valve 114d is closed. The opening and closing order of the valves 114b; 114d; 114a in step 402 is schematically illustrated in FIG. 5; and
   402b: flush system 100 via the first gas flow path 121, this step may ensure that the first gas flow tube 107 comprises the alcohol concentration inputted in step 401. In step 402b the third valve 114c is opened first, followed by the second valve 114b, and last the first valve 114a. Once the first valve 114a is opened gas is transported in the system 100 via the first gas flow path 121 and enters the test chamber 103 that has an opening 103′ to the ambient air. After a pre-determined time period the first 114a, second 114b, and third 114c valves that are open are closed again in the reverse order, i.e. the first valve 114a is closed first, followed by the second valve 114b, and last the third valve 114c is closed. The opening and closing order of the valves 114c; 114b; 114a in step 402 is schematically illustrated in FIG. 5.
403: measure alcohol concentration and possible CO₂ and water concentration with reference unit 104. To measure the alcohol and CO₂ concentration of the gas with the reference unit 104 the reference unit 104 is exposed to gas via the second gas flow path 122. To enable the measurement the fifth valve 114e is opened, followed by the second valve 114b, and last the first valve 114a. Once the first valve 114a is opened gas is transported in the system 100 via the second gas flow path 122 and the reference unit 104 is exposed to the gas for a predetermined time period of for example 2-4 seconds. After the exposure the fifth 114e, second 114b, and first 114a valve that are open are closed again in the reverse order, i.e. the first valve 114a is closed first followed by the second valve 114b, and last the fifth valve 114e. Step 403 may be repeated one or several times, so that the reference unit 104 can be exposed for gas a repeated number of times. The opening and closing order of the valves 114e; 114b; 114a in step 403 is schematically illustrated in FIG. 6a;
404: measure alcohol and possible CO₂ concentration with the at least one alcohol breath analyzer unit 111 arranged in the test chamber 103. To measure the alcohol and CO₂ concentration of the gas with the at least one alcohol breath analyzer unit 111, the at least one alcohol breath analyzer unit 111 is exposed for gas via the first gas flow path 121. To enable the measurement the third valve 114c is opened, followed by the second valve 114b, and last the first valve 114a. Once the first valve 114a is opened gas is transported in the system 100 via the first gas flow path 121 and the at least one alcohol breath analyzer unit 111 is exposed to the gas for a predetermined time period of for example 2-4 seconds. After the exposure the third 114c, second 114b, and first 114a valve that are open are closed again in the reverse order, i.e. the first valve 114a is closed first followed by the second valve 114b and last the third valve 114c. Step 404 may repeated one or several times, such as ten times for example so that the at least one alcohol breath analyzer unit 111 is exposed to gas a repeated number of times. The opening and closing order of the valves 114c; 114b; 114a in step 403 is schematically illustrated in FIG. 6b;
405: measure alcohol concentration with reference unit 104. Step 405 is a repetition of step 403. Hence, the fifth valve 114e is opened, followed by the second valve 114b, and last the first valve 114a. Once the first valve 114a is opened gas is transported in the system 100 via the third gas flow path 123 and the reference unit 104 is exposed to the gas for a predetermined time period of for example 2-4 seconds. After the exposure the fifth 114e, second 114b, and first 114a valve that are open are closed again in the reverse order, i.e. the first valve 114a is closed first followed by the second valve 114b and last the fifth valve 114e. Step 405 may repeated one or several times, such as ten times for example so that the reference unit 104 is exposed to gas a repeated number of times. The opening and closing order of the valves 114e; 114b; 114a in step 403 is schematically illustrated in FIG. 6a.

The method may comprise the steps 401 of inputting values to the system 100, 402 of flushing the system 100, 403 of measuring with the reference measuring device 104, and 404 of measuring with the alcohol breath analyzer unit 111. In such case step 403 of measuring the alcohol concentration with the reference unit 104 may be performed prior to, or after, step 404 of measuring the alcohol concentration with the at least one alcohol breath analyzer unit 111.

The reference unit 104 may comprise a reference valve 104′ that can be opened to the ambient air. In such case the reference unit 104 may flush itself in between, or during, methods of testing 400.

In case that more than one alcohol breath analyzer unit 111 are to be tested, hence that the test chamber 103 comprises more than one alcohol breath analyzer unit, the method of testing 400 may comprise an additional step 406 after step 405, in which the alcohol breath analyzer unit 111 is changed. Such a change may be performed by the device for changing alcohol breath analyzer unit 200. In a case when the method of testing 400 comprises step 406, step 406 is followed by steps 403, 404, and 405.

The method of testing 400 may comprise a step 407 in which the conditions of the testing can be changed, i.e. the relative humidity and/or temperature of the test chamber 103, and/or the concentration of alcohol. If any of the conditions are changed, step 407 is followed by step 401 and hence, the method of testing 400 start over.

Step 402 of flushing the system 100 may ensure that no residues are left in the system 100 from the previous gas concentration.

The control and registration unit 105 may be configured to control the method of testing 400 so that the control and registration unit 105 is configured to transmit signals to the valves 114a-e in the system to control when they are to be opened or closed. Step 401 of inputting alcohol concentration, temperature, and relative humidity may be performed by inputting such data to the control and registration unit 105 that may be configured to use such data to control the opening and closing of valves 114a-e in the system 100 during a method of testing 400. The control and registration unit 105 may further be configured to use such inputted data to control the relative humidity and/or temperature of the test chamber 103, as well as the temperature in the system 100 for example the temperature of the first 116a, and second 116b heater.

In all steps of the method of testing 400 when the first valve 114a is opened gas enters from the gas tank 300 into the system 100. The gas enters the liquid column 119 that comprises water and alcohol, or only water in the case that the zero level is to be tested. The concentration of alcohol in the liquid column 119 may be manually regulated or regulated using another suitable mean. When gas enters the liquid column 119 it is mixed with the liquid and a gas comprising water, air, carbon dioxide, and possible alcohol is generated in the dead space 120. The generated gas may have a controlled concentration of alcohol and/or carbon dioxide. The gas tank 300 may have an internal pressure that is higher than the internal pressure of the system 100, therefore the first valve 114a should preferably be opened last and closed first in a method where the valves 114a-e in the system 100 are opened and closed so that the first valve 114a is not open when the other valves, i.e. the second 114b, third 114c, fourth 114d, and fifth valve 114e are closed.

In one embodiment the method of testing 400 may comprise the following steps:
410: define the first 121, second 122, and third 123 gas flow path from the common starting point, i.e. the first valve 114a, to the respective endpoints, i.e. the test chamber 103 for the first gas flow path 121, the outlet 110 for the second gas flow path 122, and the reference measuring unit 104 for the third gas flow path 123. Each gas flow path 121; 122; 123 is provided with a plurality of valves, the first valve 114a is common to all gas flow paths 121; 122; 123;
411: opening the first valve 114a to provide gas to an endpoint of one of the three gas flow paths 121; 122; 123, in a sequence starting with the outermost downstream valve and consecutively open the plurality of valves in one of the first 121, second 122, or third 123 gas flow path in the upstream direction finishing with the first common valve 114a; and
412: ending the provision of gas to an endpoint by closing the valves of a gas flow path 121; 122; 123 starting with the first common valve 114a and consecutively close the plurality of valves in the gas flow path 121; 122; 123 in the downstream direction finishing with the valve closest to the endpoint, i.e. the third valve 114c for the first gas flow path 121, the fifth valve 114e for the second gas flow path 122, and the fourth valve 114d for the third gas flow path 123.

As discussed above a control and registration unit 105 is arranged to control the system 100 and to register the results from the alcohol concentration measurements. The control and registration unit 105 may record data such as power on time, startup time, CO₂ concentration, alcohol concentration, temperature, voltages, currents, etc. The control and registration unit 105 may be arranged to control the temperature of the gas generation device 101, the distribution unit 102, and the test chamber 103. The control and registration unit 105 may further be arranged to store data and generate reports that comprises temperature, relative humidity, concentration levels in the tank 300, measurement data from the alcohol breath analyzer unit 111 and reference unit 104, etc. The control and registration unit 105 may further be arranged to control the valves 114a-e inside the system 100. As described in the method of testing 400, for example in steps 402, 403, 404 and 405 above, the valves 114a-e may be controlled so that they are opened from the outside and in, i.e. the third 114c, fifth 114e, and fourth 114d valve furthest downstream the system 100 are opened first, followed by the second valve 114b in between the distribution unit 102 and the gas generation device 101, and at last the first valve 114a arranged at the gas generation device 101 is opened. Such an opening sequence of the valves 114a-e may enable the system 100 to have the same, or almost the same, pressure as the surrounding atmosphere. The valves 114a-e may further be controlled by the control and registration unit 105 so that when they are closed they are closed in the reverse order from the inside and out, hence the first valve 114a arranged at the gas generation device 101 is closed first, followed by the second valve 114b. after which one or all of the third 114c, fourth 114d, and fifth valve 114e are closed.

All aspects and embodiments can be combined with each other unless explicitly stated otherwise.

1-18. (canceled)

19. A system for testing at least one alcohol breath analyzer comprising:
an alcohol breath gas generation device comprising at least one liquid column, wherein the gas generation device comprises a gas inlet arranged at the lower part of the gas generation device and a gas outlet arranged at the upper part of the gas generation device, wherein gas enters the gas generation device at the gas inlet and exits the gas generation device at the gas outlet, and wherein the liquid in the liquid column comprises water;
a test chamber in fluid communication with the alcohol breath gas generation device, wherein the test chamber is arranged to receive at least one alcohol breath analyzer;
a reference measuring device in fluid communication with the alcohol breath gas generation device;
a control and registration unit arranged to control the parts of the alcohol breath analyzer system and to register the testing results; and
a distribution unit in fluid communication with the gas generation device, the distribution unit arranged to distribute gas from the gas generation device to one of three gas flow paths: a first gas flow path arranged to distribute gas from the gas generation device to the test chamber, a second gas flow path arranged to distribute gas from the gas generation device to the reference measuring device, and a third gas flow path arranged to distribute gas from the gas generation device to an opening to the ambient air.

20. The test system according to claim 19 wherein the system further comprises valve means arranged to shift the gas flow between the three gas flow paths: the first gas flow path, the second gas flow path, and the third gas flow path.

21. The test system according to claim 19, wherein the third gas flow path is arranged to distribute gas from the gas generation device to an outlet.

22. The test system according to any of claim 19, wherein the test system comprises a first valve arranged at the inlet at the gas generation device, arranged to regulate the gas flow into the gas generation device, a second valve arranged in between the gas generation device, and the distribution unit, the second valve is arranged to regulate the gas flow from the gas generation device to the distribution unit, a third valve, a fourth valve, and a fifth valve arranged at the distribution unit, the third valve is arranged to regulate the gas flow into the first gas tube, the fourth valve is arranged to regulate the gas flow into the third gas tube, and the fifth valve is arranged to regulate the gas flow into the second gas tube, wherein the first valve, the second valve, and the third valve are arranged to control the gas flow in the first gas flow path, the first valve, the second valve, and the fifth valve are arranged to control the gas flow in the second gas flow path, and the first valve, the second valve, and the fourth valve are arranged to regulate the gas flow in the third gas flow path.

23. The test system according to claim 22, wherein the inner diameter of the valves is 0.5-2 cm.

24. The test system according to claim 19, wherein the test system comprises at least a first and a second heater arranged to heat the system so that the temperature is higher at the distribution unit than at the gas generation device.

25. The test system according to claim 19, wherein the gas generation device has a volume of 3000-7000 cm³.

26. The test system according to claim 19, wherein the liquid column has a volume of 2000-3000 ml.

27. The test system according to claim 25, wherein the gas generation device further comprises a dead space, and wherein the volume of the dead space is 0.1-3.5 L.

28. The test system according to claim 19, wherein the test system further comprises a device for changing alcohol breath analyzer arranged in the test chamber.

29. The test system according to claim 28, wherein the device for changing alcohol breath analyzer comprises a revolver mechanism.

30. The test system according to claim 19 wherein the test system comprises more than one gas generation devices, such as two gas generation devices or three.

31. A method of testing performance of at least one alcohol breath analyzer using a system that comprises an alcohol breath gas generation device,
a test chamber in fluid communication with the alcohol breath gas generation device, the test chamber is arranged to receive at least one alcohol breath analyzer;
a reference measuring device in fluid communication with the alcohol breath gas generation device;
a control and registration unit arranged to control the parts of the alcohol breath analyzer system; and
a distribution unit in fluid communication with the gas generation device, the distribution unit arranged to distribute gas from the gas generation device to one of three gas flow paths: a first gas flow path arranged to distribute gas from the gas generation device to the test chamber, a second gas flow path arranged to distribute gas from the gas generation device to the reference measuring device, and a third gas flow path arranged to distribute gas from the gas generation device to an outlet,
wherein the method of testing comprises:
receiving input values for temperature, relative humidity, alcohol concentration to the system;
flushing the system first via the second gas flow path, and then via the first gas flow path; and
measuring, with the reference unit, the alcohol, water, and CO₂ concentration of the gas generated by the gas generation device;
wherein the at least one alcohol breath analyzer unit is arranged in the test chamber measuring alcohol, water and CO₂ concentration.

32. The method according to claim 31, wherein the system further comprises a first valve arranged at the inlet at the gas generation device, a second valve arranged in between the gas generation device and the distribution unit, a third valve, a fourth valve, and a fifth valve arranged at the distribution unit, wherein the valves are arranged to control the gas flow in the system, said method further comprising:
opening the first valve to provide gas to an endpoint of one of the three gas flow paths: the first gas flow path; the second gas flow path; or the third gas flow path, in a sequence starting with the outermost downstream valve and consecutively open the plurality of valves of one of the first gas flow path; the second gas flow path; or the third gas flow path, in the upstream direction finishing with the first common valve; and
ending the provision of gas to an endpoint by closing the valves of one of the first gas flow path; the second gas flow path; or the third gas flow path starting with the first common valve and consecutively close the plurality of valves in the flow path in the downstream direction finishing with the valve closest to the endpoint, i.e. the third valve for the first gas flow path, the fifth valve for the second gas flow path, and the fourth valve for the third gas flow path.

33. The method according to claim 31 wherein the method comprises opening of the valves in the system wherein:
one of the third valve, fourth valve, and fifth valve is opened first;
after the opening of one of the third valve, fourth valve, or fifth valve the second valve is opened; and
after the opening of the second valve the first valve is opened.

34. The method according to claim 31, wherein flushing the system comprises:
i) flushing the second gas flow path, by opening the fourth valve, followed by the second valve, and last the first valve, after a pre-determined time period the first valve is closed first, followed by the second valve, and last the fourth valve is closed; and
ii) flushing via the first gas flow path, by opening the third valve, followed by the second valve, and last the first valve, after a pre-determined time period the first valve is closed first, followed by the second valve, and last the third valve is closed; said method further comprising:
opening the fifth valve, followed by the second valve, and last the first valve, after a pre-determined time period the first valve is closed first followed by the second valve, and last the fifth valve; and
opening the third valve, followed by the second valve, and last the first valve, after a pre-determined time period the first valve is closed first followed by the second valve and last the third valve.

35. The method according to claim 31 wherein the testing on the at least one alcohol breath analyzer unit is repeated at least once.

36. The method according to claim 31 wherein the testing on the at least one alcohol breath analyzer unit is repeated ten times.
